(19) United States
(12) Patent Application Publication
(10) Pub. No.: US 2006/0155292 A1
(43) Pub. Date: Jul. 13, 2006

(54) Implant clamp and method

(76) Inventor: Jeganath Krishnan, Glenosmond (AU)

Correspondence Address: Bliss McGlynn, Suite 600, 2075 West Big Beaver Road, Troy, MI, 48084, US

(21) Appl. No.: 10/526,279

(22) PCT Filed: Sep. 19, 2003
(86) PCT No.: PCT/AU03/01229

§ 371 Date: Dec. 9, 2005

(30) Foreign Application Priority Data
Sep. 19, 2002 (AU) 2002951548

(51) Int. Cl. A61F 5/00

(52) U.S. Cl. 606/87

(57) ABSTRACT

An implant clamp for clamping an implant having a base to a bone. The clamp includes a support (10), a means to couple the support to the bone (72), an arm (16) pivotally connected to the support and an actuating member (36) operable to cause rotation of the arm relative to the support member. The arm exerts a force on the implant that is substantially perpendicular to the base.

This invention relates to a clamp for clamping an implant and to a method of fixing an implant.

In modern orthopaedic surgery various forms of implants are used. Some implants are cemented to a bone by means of a cement such as methyl methacrylate. It is desirable that the implant be held under pressure against the bone whilst the cement is cured in order to ensure that the implant is firmly bonded to the bone.

A significant problem affecting total knee replacement (TKR) is loosening of the tibial implant component, which is believed to be due to micromotion at the cement bone interface (Sherman R M P, Byrick R J, Kay J C, Sullivan T R, Waddell J P: The role of lavage in preventing haemodynamic and blood-gas changes during cemented arthroplasty. J Bone Joint Surg 65 (A):500, 1983 and Miskovsky C, Whiteside L A, White S E. The cemented unicondylar knee arthroplasty—an in vitro comparison of three cement techniques. Clin Orthop 284:215, 1992). Fixation at this interface is thought to be dependent on the penetration of cement into the trabecular bone (Miskovsky C, Whiteside L A, White S E. The cemented unicondylar knee arthroplasty—an in vitro comparison of three cement techniques. Clin Orthop 284:215, 1992 and Goldstein S A, Wilson D L, Sonstegard D A, Matthew L S. The mechanical properties of human trabecular bone as a function of metaphyseal location. J. Biomechanics 16:965, 1983), as deep penetration of cancellous bone by cement has been shown to reduce micromotion and posterior lift-off and to be important in achieving durable rigid fixation (Miskovsky C, Whiteside L A, White S E. The cemented unicondylar knee arthroplasty—an in vitro comparison of three cement techniques. Clin Orthop 284:215, 1992).

Cement penetration into bone is multifactorial. It is directly proportional to pore diameter and the square root of the pressure applied to the cement, and inversely proportional to time from initial mixing of the cement (Walker P S, Soudry M, Ewald F C, McVickar H. Control of cement penetration in total knee arthroplasty. Clin Orthop 185:155, 1984). Improved penetration of cement is also achieved by the use of high volume, high pressure pulsatile lavage to clean the cut surface, low cement viscosity (Walker P S, Soudry M, Ewald F C, McVickar H. Control of cement penetration in total knee arthroplasty. Clin Orthop 185:155, 1984, Krause W R, Krug W, Eng B, Miller J. Strength of the cement-bone interface. Clin Orthop 163:290, 1982 and Ritter M A, Herbst S A, Keating E M, Faris P M. Radiolucency at the bone-cement interface in total knee replacement. J Bone Joint Surg 76(A):60, 1994), and increased time that pressure is applied to the cement (Walker P S, Soudry M, Ewald F C, McVickar H. Control of cement penetration in total knee arthroplasty. Clin Orthop 185:155, 1984). Penetration of cement is decreased in sclerotic bone (Kim Y, Walker P S, Deland J T. A cement impactor for uniform cement penetration in the upper tibia. Clin Orthop 182:206, 1984), and peripheral penetration is decreased by cement leakage around the edges of the prosthesis (Walker P S, Soudry M, Ewald F C, McVickar H. Control of cement penetration in total knee arthroplasty. Clin Orthop 185:155, 1984, Kim Y, Walker P S, Deland J T. A cement impactor for uniform cement penetration in the upper tibia. Clin Orthop 182:206, 1984 and Vertullo C J, Davey J R. The effect of a tibial baseplate undersurface peripheral lip on cement penetration in total knee arthroplasty. J. Arthroplasty 16:487, 2001).

Less than 1 to 2 mm of penetration results in low cement-bone interface strength and predisposes to micromotion (Kim Y, Walker P S, Deland J T. A cement impactor for uniform cement penetration in the upper tibia. Clin Orthop 182:206, 1984 and Bert J M, McShane M. Is it necessary to cement the tibial stem in cemented total knee arthroplasty? Clin Orthop 356:73, 1998) and radiological evidence of loosening (Walker P S, Soudry M, Ewald F C, McVickar H. Control of cement penetration in total knee arthroplasty. Clin Orthop 185:155, 1984). Increased cement penetration has been shown to increase tensile and shear strength of the cement bone interface (Walker P S, Soudry M, Ewald F C, McVickar H. Control of cement penetration in total knee arthroplasty. Clin Orthop 185:155, 1984 and Ritter M A, Herbst S A, Keating E M, Faris P M. Radiolucency at the bone-cement interface in total knee replacement. J Bone Joint Surg 76(A):60, 1994), however, greater than 5 mm of penetration results in heat necrosis of bone, does not increase the strength of the interface and results in increased loss of bone stock if revision arthroplasty is required (Walker P S, Soudry M, Ewald F C, McVickar H. Control of cement penetration in total knee arthroplasty. Clin Orthop 185:155, 1984 and Kim Y, Walker P S, Deland J T. A cement impactor for uniform cement penetration in the upper tibia. Clin Orthop 182:206, 1984). The ideal is 3- to 5-mm of uniform penetration (Walker P S, Soudry M, Ewald F C, McVickar H. Control of cement penetration in total knee arthroplasty. Clin Orthop 185:155, 1984 and Kim Y, Walker P S, Deland J T. A cement impactor for uniform cement penetration in the upper tibia. Clin Orthop 182:206, 1984).

The use of clamp fixation in pressurization of cement in the proximal tibia has been previously described (Kanekasu K, Yamakado K, Hayashi H. The clamp fixation method in cemented total knee arthroplasty. Dynamic experimental and radiographic studies of the tibial baseplate clamper. Bull Hosp Jt Dis 56:218, 1997). This method provides large initial pressure, a lack of variance in force over time and deep cement penetration. The method also allows the surgeon to concurrently cement the other components. The clamp in this arrangement is a generally scissor action device wherein one of the tips thereof can penetrate the bone adjacent to a site where the implant is to be mounted. The other tip is provided with an implant engaging member. The device includes a screw clamp to force the handles of the scissors apart and thus force the implant engaging member into the implant. The device is somewhat complicated and has the disadvantage that the force exerted on the implant would include components at an angle to the normal and this may cause unwanted lateral displacement of the implant. Also, there is no provision for adjustability of the point of contact of the implant engaging member with the implant.

The tensile and shear strengths of the cement bone interface have been assessed by previous authors (Walker P S, Soudry M, Ewald F C, McVickar H. Control of cement penetration in total knee arthroplasty. Clin Orthop 185:155, 1984 and Ritter M A, Herbst S A, Keating E M, Faris P M. Radiolucency at the bone-cement interface in total knee replacement. J Bone Joint Surg 76 (A):60, 1994), although their relevance to the clinical setting has not been established as tensile and shear stresses that may pull the tibial component from the bone are not commonly experienced by a TKR in situ. However, it has been postulated that increasing the tensile and shear strength of the cement bone interface should decrease micromotion (Walker P S, Soudry M, Ewald F C, McVickar H. Control of cement penetration in total knee arthroplasty. Clin Orthop 185:155, 1984 and Ritter M A, Herbst S A, Keating E M, Faris P M. Radiolucency at the bone-cement interface in total knee replacement. J Bone Joint Surg 76 (A):60, 1994). Micromotion is a microscopic rocking of an implant on the cut tibial surface, with depression of one side of the implant and elevation of the contralateral side. The stiffness of the cement-bone composite that is being compressed is therefore likely to be relevant to micromotion and loosening of the tibial component. This is supported by evidence that there is increased subsidence of uncemented tibial components in tibiae with low bone mineral density (BMD), although the relationship with BMD is eliminated by the use of cement (Li M G, Nilsson K G. The effect of preoperative bone quality on the fixation of the tibial component in total knee arthroplasty. J Arthroplasty 15:744, 2000).

The stiffness of trabecular bone in normal, osteoarthritic, and rheumatoid knees has previously been examined (Finlay J B, Bourne R B, Kraemer W J, Moroz T K, Rorabeck C H. Stiffness of bone underlying tibial plateaus of osteoarthritic and normal knees. Clin Orthop 247:193, 1989 and Yang J P, Bogoch E R, Woodside T D, Hearn T C. Stiffness of trabecular bone of the tibial plateau in patients with rheumatoid arthritis of the knee. J Arthroplasty 12:798, 1997). The stiffness of the cement-bone composite has not been previously reported.

The object of the present invention is to provide a novel clamp which can be used for clamping an implant during curing of the cement. The same device can also be used to pressurise the cement mantle to allow deeper and more uniform penetration of cement through the bone tissue.

According to the present invention there is provided an implant clamp for clamping an implant having a base to a bone, said clamp including a support member, means for coupling the support member to a bone, an arm pivotally connected to the support member, and an actuating member which is operable to cause rotation of the arm relative to the support member whereby the arm exerts a force on the implant which is substantially perpendicular to the base of the implant.

The implant defined above has the advantage that it minimises the possibility of lateral movement of the implant during clamping because the clamping force is essentially limited to forces which are perpendicular to the base of the implant.

Preferably, the means for coupling includes one or more removable pins which pass through bores in the support member.

Preferably further, the actuating member comprises a manually operable screw acting between the arm and the support member.

Preferably further, the support means is generally in the form of a plate.

Preferably further, a number of bores are provided through the plate so that a plurality of said pins can be used to securely connect the support member to a bone.

Preferably further, an implant engaging member is mounted on an end of the arm in order to engage the implant.

The invention also provides a method of fixing an implant to a bone comprising the steps of preparing a surface of the bone for receipt of an implant, coupling a clamping member to the bone adjacent to said surface, applying implant cement between the surface and the implant, actuating the clamping member so that the implant is forced into engagement with the cement on the surface by means of an engagement member which asserts a force on the implant substantially solely in a direction which is perpendicular to said surface of the bone.

Preferably the method includes the step of curing the cement whilst the clamp is still operative.

Preferably further, the cement is cured with ultraviolet radiation.

The invention also provides an implant clamp for clamping an implant to a bone, the clamp including:
a support member;
connecting means for connecting the support member to the bone adjacent to a site where the implant is to be mounted;
an arm;
adjustable pivot means for forming a pivotal connection between the arm and the support member about one or more selectable pivot axes; and
an actuating member which is operable to cause rotation of the arm relative to the support member whereby the arm exerts a force on the implant at a location thereon which depends on which of the selectable pivot axes is selected.

Preferably, the adjustable pivot means includes a pivot shaft, first spaced pivot holes in the support member and second spaced pivot holes in the arm, the arrangement being such that one of the selectable pivot axes can be selected by aligning one of the first pivot holes with one of the second holes and passing the pivot shaft through said aligned holes.

The arrangement defined above has the advantage that the pivot axis can be selected so that the arm exerts a force generally at the centre of the implant. This results in better fixation of the implant compared to arrangements in which the clamping force is offset from the centre of the implant.

The invention also provides an implant clamp for clamping an implant to a bone, the clamp including:
a support member having a plurality of connecting holes therethrough;
mounting pins which can pass through the connecting holes to penetrate the bone for mounting the support member adjacent to a site where the implant is to be mounted;
an arm pivotally connected to the support member; and
an actuating member which is operable to cause rotation of the arm relative to the support member whereby the arm exerts pressure on an implant in order to force the implant into firm contact with the bone at said site.

This arrangement has the advantage that in use the support member acts as a fixed pivot post to which the arm is pivotally connected so that the arm can then apply a force to the implant such that the pressure between the implant and the bone is generally uniform.

The invention will now be further described with reference to the accompanying drawings, in which:

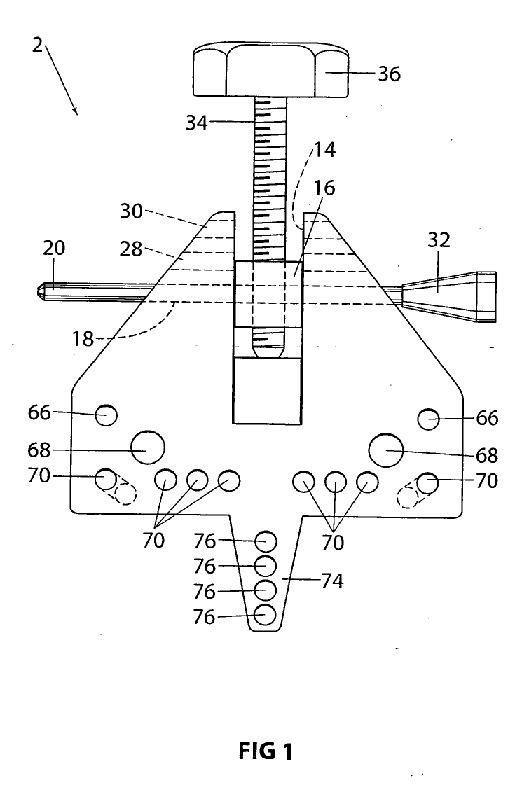

FIG. 1 is a frontal view of an implant clamp;

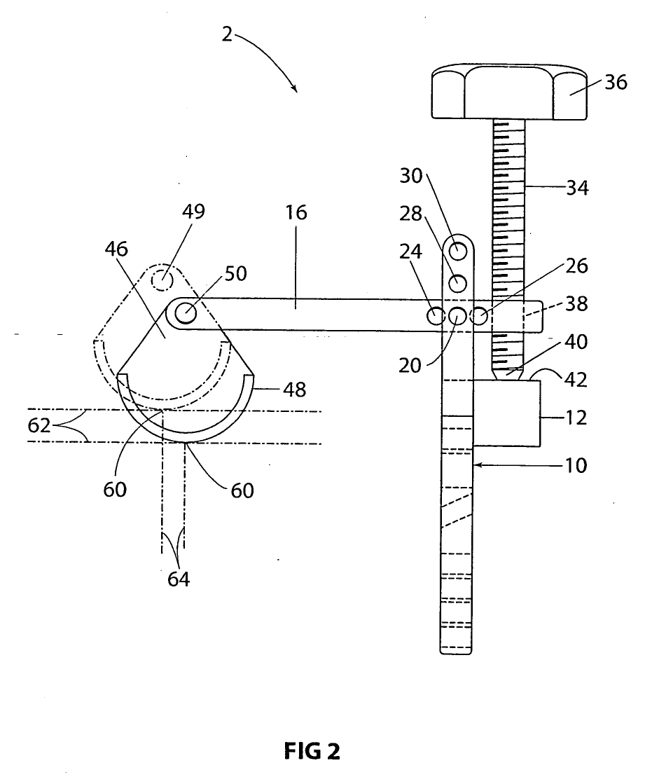

FIG. 2 is a side view of the implant clamp;

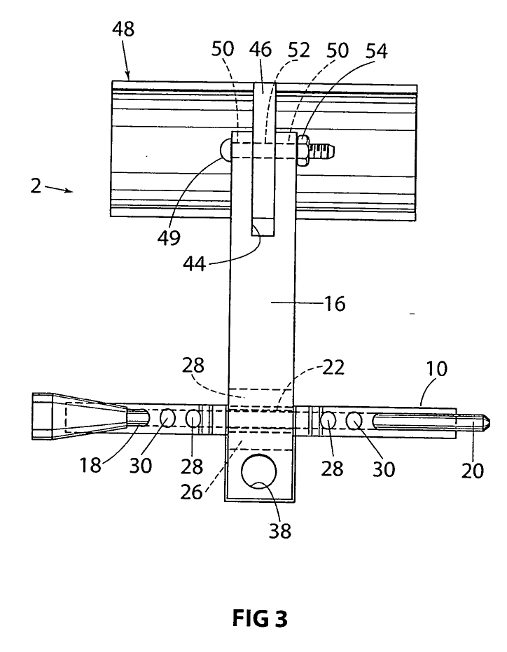

FIG. 3 is a plan view of the implant clamp;

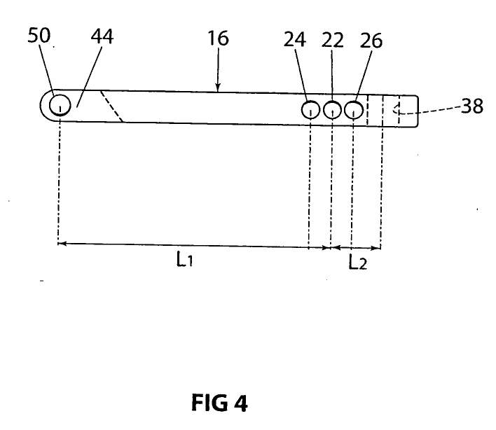

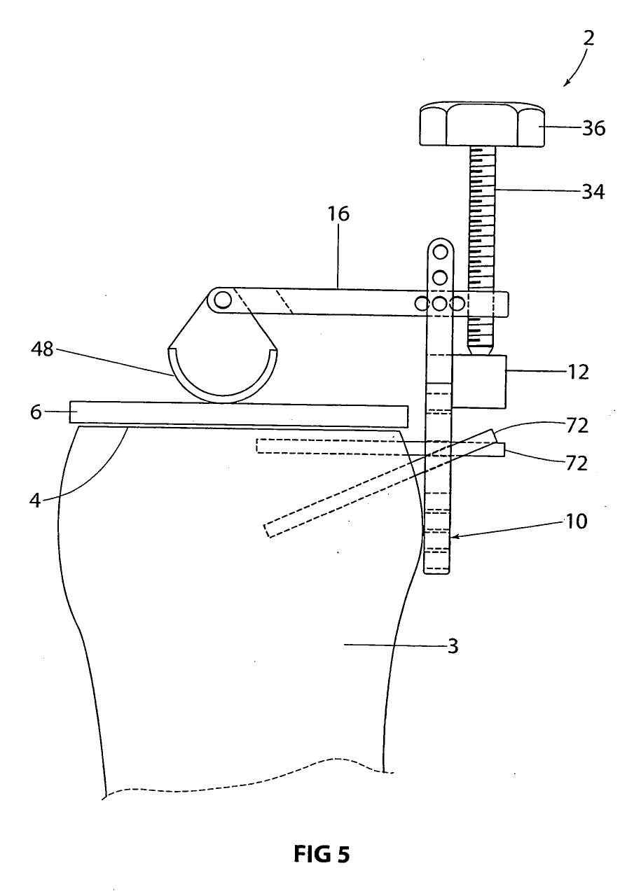

FIG. 4 is a side view of the implant clamp arm; and
FIG. 5 schematically illustrates the use of the implant clamp.

FIGS. 1 to 4 illustrate an implant clamp 2 constructed in accordance with the invention. The clamp is particularly suited for use in knee replacement surgery. In that surgery, the top part of the tibia 3, i.e. the articular surface, is removed so as to define a flat bone surface 4 against which an implant 6 can be cemented in position. The clamp 2 of the invention can be used to clamp the implant 6 against the surface 4 during curing of the implant cement.

The clamp 2 of the invention is made from a number of components which are arranged for easy disassembly for cleaning and sterilisation. It is preferred that the components are also made from stainless steel so that they can be autoclaved. Alternatively, the components could be injection moulded from a suitable polymer or a polymer having hardening agents added thereto.

The clamp 2 comprises a support plate 10 from which projects an abutment block 12. Above the block 12 is a slot 14 which receives a clamp arm 16. The plate 10 includes a bore 18 through which a pivot shaft 20 passes. The pivot shaft 20 also passes through a hole 22 in the arm 16. As best seen in FIG. 2, the arm 16 may include a number of adjacent holes 24 and 26, as shown in FIG. 2, so as to provide adjustment of the pivot point of the arm 16 relative to the plate 10. Similarly, the plate 10 itself may include bores 28 and 30 to permit vertical adjustment of the pivot point of the arm 16 relative to the plate 10. As best shown in FIGS. 1 and 3, the end of the shaft 20 includes a tapered head 32 which assists in holding the shaft 20 in the selected bore through the plate 10.

The clamp includes a screw 34 having a head 36 which is relatively large so that it can be manually operated. The head 36 may include peripheral grooves or scallops so as to make it easier to grip manually or to grip with pliers or the like. The screw 34 passes through a threaded bore 38 in the arm 16 so that the lower end 40 of the screw abuts the top face 42 of the block 12.

In use, rotation of the screw 34 in the threaded bore 38 causes rotation of the arm 16 relative to the plate 10. The free end of the arm 16 is bifurcated at 44 and receives a mounting plate 46 of a pressure applying member 48. The member 48 may comprise a semi-cylindrical shell having a diameter say of about 20 mm and a length of say 50 mm. The mounting plate 46 is preferably located at the centre of the member 48, as best seen in FIG. 3. The mounting plate 46 is adjustably mounted relative to the arm 16 by means of a mounting bolt 49 which passes through aligned bores 50 and 52 in the arm 16 and plate 46 respectively. A nut 54 is used to fix the position of the mounting plate 46 relative to the arm 16. Because the pressure applying member 48 is cylindrical in shape and is pivotally connected to the end of the arm 16, the member 48 is effective to transmit only forces which are essentially perpendicular to the bone surface 4. This avoids the application of components of force to the implant 6 which would be in the direction of the plane of the surface 4 of the bone, which lateral forces may tend to cause displacement of the implant.

As will be appreciated from FIG. 2, alteration of the member 48 relative to the arm 16 can be used to change the position of the lowermost point 60 on the member 48 relative to the plate 10 both vertically and horizontally as indicated by lines 62 and 64. As will be explained below, the lowermost point 60 constitutes the point of contact with the upper surface of the implant 6. This enables fine adjustment of the position of the point of contact on the implant to enable it to be correctly aligned with the flat surface 4 of the tibia. In the illustrated arrangement, the pressure applying member 48 is cylindrical in shape and therefore the lowermost point 60 will define a line of contact with the upper surface of the implant 6. The arrangement of the invention which enables adjustment both horizontally and vertically of the line of contact of the force applying member 48 with the upper surface of the implant enables the user to select the line of contact so that it is generally centrally located on the implant. This avoids uneven application of force to the implant and therefore generally ensures that the base surface of the implant is brought into intimate contact with the surface 4 of the bone so as to avoid any angular misalignment therewith.

The plate 10 includes upper bores 66, intermediate bores 68 and lower bores 70. The bores are for receipt of arresting pins 72 which pass therethrough and are temporarily located in bores drilled in the tibia, as shown in FIG. 4. It is preferred that the outermost lower bores 70 are inclined downwardly and inwardly at an angle of about 20° so as to provide a better mechanical fixing of the plate 10 to the tibia. The lower edge of the plate 10 includes a downward extension 74 which is provided with a plurality of central bores 76 for receipt of additional mounting pins if these are required. The pins for mounting the plate to the tibia can be provided with heads or bent ends (not shown) which are engagable with the outer side of the plate to firmly retain the plate against the tibia.

FIG. 4 shows the clamp arm 16 in more detail. In this drawing the centres of the various holes and bores are marked with broken lines. The arm 16 essentially functions as a lever. The overall length of the arm 16 is say 45 mm and the spacing between the bores 50 and 38 is 37 mm. The centres of the holes 26, 22 and 24 are spaced from the centre line of the bore 38 by the distances of 5 mm, 9 mm and 13 mm respectively.

When the shaft 20 is selected to pass through the hole 22, the effective arm ratio L1/L2 is about 6.4. The effective lengths L1 and L2 can of course be varied by selecting the shaft 20 to pass through the holes 26 and 24 as required. Table 1 below sets out the effective arm ratio in accordance with the hole selected as the fulcrum hole for the clamp arm.

TABLE 1

| Fulcrum Hole | L1 | L2 | L1/L2 |
|---|---|---|---|
| 26 | 32 | 5 | 6.4 |
| 22 | 28 | 9 | 3.1 |
| 24 | 24 | 13 | 1.8 |

The clamp of the invention can be made to suit various requirements. When it is used for implantation of an artificial knee joint, the clamp plate 10 may have a height of about 50 mm, a width of about 60 mm and a thickness of 5 mm. As mentioned above, all of the components are preferably stainless steel so that it can be readily autoclaved.

In use of the device of the invention, the plate 10 is first securely fixed to the upper part of the tibia by the pins 72 as shown in FIG. 5. Normally about four pins would be required for this purpose. The arm 16 can then be placed in position in the slot 14 and the shaft 20 can be located so as to form the pivotal connection. A layer of cement can then be applied to the bone surface 4, a cement spreader plate (not shown) can be located on top of the layer of cement. The clamp can then be operated by screwing down the screw 36 so that the member 48 engages the cement spreader plate. This causes squeezing of the cement into porous parts of the bone so as to ensure good contact therewith. Any excess cement can be removed from the side of the bone surface 4. After a few minutes to permit some curing of the cement, the clamp can then be unscrewed so that the cement spreader plate can be removed. The implant 6 (the tibial base plate) can then be located on the cement in its correct position relative to the tibia and then impacted with a hammer. The surgeon then operates the screw head 36 so as to apply a controlled downward force on the upper face of the implant 6 by appropriate adjustment of the pivotal mounting of the arm 16 relative to the plate and of the member 48 relative to the free end of the arm 16 the exact location of the point of contact of the member 48 on the top surface of the implant 6 can be finely adjusted. Also, the screw head 36 enables the amount of downward force to be controlled. With the implant 6 firmly held in its correct position, ultraviolet radiation can then be used to cure the cement. After curing of the cement, the screw 34 is operated so that the member 48 disengages the implant 6. The clamp can then be removed from the tibia 3 by removal of the pins which hold the plate 10 to the tibia.

A prototype of the invention has been tested in order to determine the efficacy thereof The tests have been carried out on porcine tibiae and these results are indicative of good performance in human patients. Some details of the porcine testing are set out below.

Twenty cadaveric juvenile porcine tibiae were randomly allocated into four groups. The tibiae were cut through the metaphysis using an oscillating saw. The metaphyseal bone was analysed using bone densitometry (DEXA, Lunar Prodigy Bone Densitometer, GE Lunar Corporation, Madison, Wis., USA) to ensure uniformity of the four groups. The cut surface was then prepared with high pressure, high volume pulsatile lavage with normal saline. Polymethylmethacrylate cement (Surgical Simplex200 , Stryker Howmedica Osteonics, Allendale N.J., USA) was mixed according to the manufacturer's instructions at the ambient temperature of 22° C. and applied 3 minutes after mixing. A 10 cm×10 cm×0.5 cm square, flat, cold-worked piece of stainless steel was used to simulate a prosthesis.

One group (digital) underwent packing of the cement into the cut surface with digital pressure. As second group (undersurface) underwent coating the undersurface of the model prosthesis with cement. In both groups the model prosthesis was then impacted with a mallet and manual pressure applied until the cement had cured. For a third group (pressuriser) cement was first applied to the cut tibial surface, the model prosthesis was then impacted and pressure applied with the implant clamp 2 of the invention. The control group received no cement or implant.

The specimens were stored in saline-soaked towels and frozen at −30° C. Computed tomography (CT) scans were then performed using a GE High Speed Advantage CT system (General Electric, Milwaukee Wis., USA), with one millimetre slices taken parallel to the cut surface of the tibia. The digital CT images were then analysed to a depth of 5 mm using digital image analysis software (IDL Version 4, Research Systems Inc., Boulder Colo., USA). A manual trace was performed inside the cortical rim of each slice to exclude cortical bone and soft tissue from the analysis. The penetration of cement into the cancellous metaphyseal bone was calculated as a percentage of pixels within the manual trace having a grey scale greater than 150. This level was chosen as the threshold because it was observed that scans through metaphyseal trabecular bone with no cement recorded zero pixels with grey scale greater than 150. To assess the intra-observer variability, this was repeated ten times at a depth of 2 mm on one specimen chosen at random, in this case the fourth specimen from the undersurface group.

The tibiae were then thawed and cut 1.5 cm distal to the cut surface and mounted for indentation testing. Specimens were placed face down on a clean latex covered stainless steel plate and encircled by a 2-cm high stainless steel cylindrical mould. This mould was filled with polymethylmethacrylate cement and allowed to set. The specimen was inverted and the tibial plateau, embedded horizontally in the cement, examined. A line was drawn across the plateau between points defining the maximum mediolateral width of the specimen. This line was divided in thirds by perpendicular lines creating six regions. The six regions reported were anteromedial (AM), anterior intercondylar (AI), anterolateral (AL), posteromedial (PM), posterior intercondylar (PI) and posterolateral (PL). The flat end of a cylindrical 4-mm diameter indenter was fixed to the actuator of an Instron model 8511 servohydraulic materials testing machine (Instron Pty Ltd, High Wycombe, UK). Indentation tests were performed to a depth of 0.5 mm at a rate of 2 mm/min. Four tests were performed in each region. Stiffness, defined as the slope of the linear region of the loading curve, was calculated. Statistical analysis was performed using univariate and repeated measures analysis of variance (ANOVA) and Tukey's Honestly Significant Difference post-hoc tests.

The mean BMD of the tibiae was 0.88 g/cm2 (SD 0.40) and no significant difference in BMD was seen between the groups (Univariate ANOVA, P=0.317).

Assessment of the intra-observer variability for determination of cement penetration gave a coefficient of variation of 0.66 (mean 27.6%, SD 0.18%). The cement penetration data are summarised in Table 2.

TABLE 2
Cement penetration expressed as a percentage of
total surface area (mean; SD).

| | Depth | | | | |
|---|---|---|---|---|---|
| Group | 1 mm | 2 mm | 3 mm | 4 mm | 5 mm |
| Finger Packed | 55 (14) | 42 (9) | 14 (4) | 4 (2) | 3 (2) |
| Undersurface | 78 (8) | 46 (11) | 21 (10) | 7 (4) | 4 (4) |
| Clamp 2 | 64 (6) | 43 (19) | 15 (8) | 6 (4) | 5 (5) |

Independent ANOVAs were performed for each depth. Cement penetration at 1 mm was significantly greater with coating the undersurface of the prosthesis with cement than with finger packing (P=0.008). There was no significant difference at deeper levels or between the implant clamp 2 of the invention and either of the two other groups at any level (P>0.3 in all cases).

The surface stiffness results are summarised in Table 3.

TABLE 3
Mean stiffness and standard deviations (N/mm)
for each group and region.

| | Region | | | | | | | |
|---|---|---|---|---|---|---|---|---|
| | | | | | | | | Effect |
| | | | | | | | | of |
| Group | AM | AI | AL | PL | PI | PM | Global | Region¹ |
| Control | 623 | 591 | 1104 | 1202 | 922 | 939 | 897 | <0.001* |
| | (199) | (319) | (454) | (467) | (531) | (296) | (441) | |
| Finger | 1438 | 1235 | 1291 | 1416 | 1566 | 1640 | 1431 | 0.019* |
| Packed | (422) | (378) | (464) | (337) | (421) | (435) | (427) | |
| Under- | 1941 | 1771 | 2058 | 2139 | 2228 | 2153 | 2038 | 0.038* |
| surface | (458) | (383) | (475) | (536) | (521) | (617) | (517) | |
| Clamp 2 | 1525 | 1316 | 1425 | 1367 | 1425 | 1357 | 1471 | 0.621 |
| | (549) | (528) | (519) | (410) | (291) | (330) | (458) | |

¹Univariate ANOVA. AM, anteromedial: AI, anterior intercondylar: AL, anterolateral: PM, posteromedial: PI, posterior intercondylar: PL, posterolateral.

The mean global stiffness of all cemented groups was significantly greater than for the control group (P<0.001). Coating the undersurface of the prosthesis with cement also produced greater mean global stiffness than the other two cementing techniques (P<0.001). Significant differences were found between different regions in the control group (P<0.001), finger packing the cement (P=0.019) and coating the undersurface of the prosthesis with cement (P=0.038). Such differences were not found using the clamp of the invention (P=0.621).

Cement penetration of the tibial metaphysis was significantly affected by the method of pressurisation of the cement. Coating the undersurface of the prosthesis with cement followed by impaction with a hammer produced significantly greater cement penetration at a depth of one millimetre than finger packing of the cut tibial surface. No significant differences were shown between the clamp 2 of the invention and the other two techniques. The stiffness of the cement-bone composite to axial loading is also significantly affected by the cementing technique. Undercoating of the prosthesis and impaction produced the most consistent increase in stiffness of the cement bone composite, which is in keeping with the results of cement penetration.

Regional differences in surface stiffness were found in the control tibiae that were not cemented. The clamp 2 of the invention was the only method of cementing which eliminated these regional differences. This more uniform stiffness across the tibial plateau may reduce micromotion and loosening of the tibial component after total knee arthroplasty.

The mean BMD of the tibial metaphysis of a population undergoing TKR has been reported previously as 0.81 g/cm2 (range, 0.15-1.33 g/cm2) (11), which is similar to that found in the porcine tibiae of this study. Mean stiffness of different regions of the cut surface of the tibia from patients undergoing TKR for osteoarthritis shows wide variation, ranging from 586 N/mm (SD, 203 N/mm) to 1786 N/mm (SD, 807 N/mm) (12). The stiffness of the control porcine tibiae fall within this range (Table 3). These two properties support the validity of the porcine model used in the test results.

When mounting the tibiae for indentation testing it was necessary to hold the cement surrounding the sample with clamps, and where there was an uneven base this was supported with washers. It is possible that some settling occurred at the site of these supports during testing. There may also be some error in the angle of indentation, despite efforts to ensure it was perpendicular to the tested area. These potential errors would not have favoured any particular group.

The use of cement increases the stiffness of the cancellous bone and should reduce micromotion and the incidence of aseptic loosening of tibial base plates in TKR. The cementing technique should aim for consistent stiffness across the cement bone composite. In this study maximum penetration was achieved using undercoating of the prosthesis and impaction with a mallet, however this method also produced regional variations in stiffness. The most uniform stiffness was achieved by the use of the clamp 2 of the invention. The use of the clamp of the invention also leaves the surgeons' hands free to work on the femur during tibial cement curing.

Many modifications will be apparent to those skilled in the art without departing from the spirit and scope of the invention.

1. An implant clamp for clamping an implant having a base to a bone, said clamp including a support member, means for coupling the support member to a bone, an arm pivotally connected to the support member, and an actuating member which is operable to cause rotation of the arm relative to the support member whereby the arm exerts a force on the implant which is substantially perpendicular to the base of the implant.

2. An implant clamp as claimed in claim 1 wherein the actuating member comprises a manually operable screw acting between the arm and the support member.

3. An implant clamp as claimed in claim 2 wherein the support means is generally in the form of a plate.

4. An implant clamp as claimed in claim 3 wherein the means for coupling includes one or more removable pins which pass through bores in the support member.

5. An implant clamp as claimed in claim 4 wherein a number of bores are provided through the plate so that a plurality of said pins can be used to securely connect the support member to a bone.

6. An implant clamp as claimed in claim 3 wherein the plate includes a slot and wherein said arm extends transversely through the slot and wherein the arm is pivotally connected to the plate by means of a removable shaft which is received within aligned bores in the plate so that the arm functions as a lever.

7. An implant clamp as claimed in claim 6 wherein the screw passes through a threaded bore in one end of the arm, one end of the screw being engagable with a block which projects from the plate.

8. An implant clamp as claimed in claim 7 wherein the other end of the screw is provided with a head for manually rotating the screw.

9. An implant clamp as claimed in claim 7 wherein an implant engaging member is mounted on the other end of the arm for engaging, in use, the implant.

10. An implant clamp as claimed in claim 9 wherein the implant engaging member is pivotally connected to the other end of the arm for limited rotation about an axis which is parallel with said shaft.

11. An implant clamp as claimed in claim 10 wherein the implant engaging member includes a part cylindrical surface.

12. An implant clamp as claimed in claim 11 including a mounting plate from which the part cylindrical surface extends.

13. An implant clamp as claimed in claim 12 wherein said other end of the arm includes a slot and said mounting plate is located in the slot.

14. An implant clamp as claimed in claim 11, wherein the diameter of said part cylindrical surface is about 20 mm.

15. An implant clamp as claimed in claim 11, wherein the length of said part cylindrical surface is about 50 mm.

16. An implant clamp as claimed in claim 6 wherein the plate has a plurality of aligned bores so that the shaft can be selectively placed through the bores to thereby adjust the position of the arm relative to the plate.

17. An implant clamp as claimed in claim 16 wherein the arm includes a plurality of holes through which the shaft may selectively pass to thereby effectively vary the lever ratio of said arm.

18. An implant clamp as claimed in claim 17 wherein there are three of bores in the plate and three of the holes in the arm.

19. An implant clamp as claimed in claim 17 wherein the lever ratio of said arm is variable in the range from about 1.5 to 7.

20. An implant clamp as claimed in claim 19 wherein the lever ratio of said arm is 1.8, 3.1 or 6.4.

21. An implant clamp as claimed in claim 1 adapted for use in total knee replacement surgery and wherein said bone is the tibia of a patient.

22. A method of fixing an implant to a bone comprising the steps of preparing a surface of the bone for receipt of an implant, coupling a clamping member to the bone adjacent to said surface, applying implant cement between the surface and the implant, actuating the clamping member so that the implant is forced into engagement with the cement on the surface by means of an engagement member which asserts a force on the implant substantially solely in a direction which is perpendicular to said surface of the bone.

23. A method as claimed in claim 22 wherein the clamping member comprises an implant clamp as claimed in any one of claims 1 to 20.

24. A method as claimed in claim 23 wherein the implant is a knee prosthesis and the bone is the tibia of a patient.

25. An implant clamp for clamping an implant to a bone, the clamp including:
a support member;
connecting means for connecting the support member to the bone adjacent to a site where the implant is to be mounted;
an arm;
adjustable pivot means for forming a pivotal connection between the arm and the support member about one or more selectable pivot axes; and
an actuating member which is operable to cause rotation of the arm relative to the support member whereby the arm exerts a force on the implant at a location thereon which depends on which of the selectable pivot axes is selected.

26. An implant clamp as claimed in claim 27 wherein the adjustable pivot means includes a pivot shaft, first spaced pivot holes in the support member and second spaced pivot holes in the arm, the arrangement being such that one of the selectable pivot axes can be selected by aligning one of the first pivot holes with one of the second holes and passing the pivot shaft through said aligned holes.

27. An implant clamp for clamping an implant to a bone, the clamp including:
a support member having a plurality of connecting holes therethrough;
mounting pins which can pass through the connecting holes to penetrate the bone for mounting the support member adjacent to a site where the implant is to be mounted;
an arm pivotally connected to the support member; and
an actuating member which is operable to cause rotation of the arm relative to the support member whereby the arm exerts pressure on an implant in order to force the implant into firm contact with the bone at said site.
